(19) United States
(12) Patent Application Publication
(10) Pub. No.: US 2024/0226057 A1
(43) Pub. Date: Jul. 11, 2024

(54) NEW USE OF MONENSIN

(71) Applicant: MAST PHARMA AB, UPPSALA (SE)

(72) Inventors: Gunnar PEJLER, Uppsala (SE); Aida PAIVANDY, Uppsala (SE); Marco MACCARANA, Uppsala (SE); Mikael ADNER, Uppsala (SE)

(21) Appl. No.: 18/573,570

(22) PCT Filed: Jun. 10, 2022
(86) PCT No.: PCT/SE2022/050570

§ 371 Date: Dec. 22, 2023

(30) Foreign Application Priority Data
Jun. 28, 2021 (SE) 2150825-4

(51) Int. Cl. A61K 31/352; A61K 45/06; A61P 37/02

(52) U.S. Cl. CPC A61K 31/352; A61K 45/06; A61P 37/02

(57) ABSTRACT

The present invention relates to the compound monensin as a potent agent or drug for dampening the harmful activities of mast cells and eosinophils, and use of the same in the alleviation, treatment and/or prevention of mast cell- or eosinophil-dependent pathologies, such as inflammatory diseases.

### TECHNICAL FIELD

The present disclosure relates to monensin, an antibiotic isolated from Streptomyces cinnamonensis. Monensin was identified to be a selective and potent inducer of apoptosis in granulocytes comprising acidic cytoplasmic granules. Further, the invention relates to a pharmaceutical composition comprising monensin or its pharmaceutically acceptable salt for use in a treatment of diseases or conditions wherein said granulocytes have a detrimental impact, and a method of treatment.

### BACKGROUND

Inflammatory diseases include a vast array of disorders and conditions that are characterized by inflammation, examples include allergy, asthma, anaphylaxis, skin diseases, autoimmune diseases, coeliac disease, glomerulonephritis, hepatitis, inflammatory bowel disease, preperfusion injury and transplant rejection.

Allergic diseases have increased in the Western world over the past 2-3 decades. For example, asthma affected an estimated 262 million people in 2019 and caused 461000 deaths (Global burden of 369 diseases and injuries in 204 countries and territories, 1990-2019: a systematic analysis for the Global Burden of Disease Study 2019. Lancet. 2020;396(10258): 1204-22).

Granulocytes are a type of white blood cells that have a role in both the innate and adaptive immune system. They are characterized by having granules in their cytoplasm which are released during infections, allergic reactions, and asthma, a process called degranulation.

There are four types of granulocytes; basophils, eosinophils, neutrophils and mast cells. The focus of the present disclosure resides in the granulocytes having acidic cytoplasmic granules i.e eosinophils and mast cells.

The mast cell granules contain an array of preformed bioactive compounds, including histamine, proteoglycans of serglycin type, cytokines, growth factors and various proteases. When mast cells are activated, either through IgE-mediated- or other mechanisms, these preformed mediators are released. Mast cell activation leads to de novo synthesis and releases of a range of additional inflammatory mediators such as prostaglandins, leukotrienes, cytokines, chemokines and growth factors. Strategies to dampen the detrimental impact of mast cells on such conditions include the use of drugs targeting individual mast cell mediators, such as antihistamines, or strategies to prevent mast cell degranulation. The latter include the use of various mast cell stabilizers such as ketotifen and chromoglycate, but also anti-IgE therapy.

The eosinophil granules contain many chemical mediators, such as eosinophil peroxidase, ribonuclease (RNase), deoxyribonucleases (DNase), lipase, plasminogen, and major basic protein, which are released by degranulation following activation of the eosinophil, and are toxic to both parasite and host tissues. Strategies to dampen the detrimental impact of eosinophils on such conditions include monoclonal antibody therapy and antagonists of leukotriene synthesis or receptors.

In agreement with their massive capacity for secreting pro-inflammatory mediators, mast cells and eosinophils are known to have a major impact on several pathological settings of inflammatory nature. Most notably, these cells are strongly implicated in allergic responses including asthma. These cells are also involved in urticaria, and implicated in a range of additional pathologies, such as eosinophilic esophagitis, fibrosis and cancer.

However, all these strategies have their limitations. For example, antihistamines will only block effects mediated by histamine but will not block the effects of other components released by mast cells. Mast cell stabilizers have limited efficacy and will not prevent the effects of components that are released from mast cells independently of degranulation. Anti-IgE therapy, although showing high efficacy under certain settings and in selected patients, is costly and is not effective for all patients.

Altogether, mast cell and eosinophil activation can result in the release of an impressive array of potent bioactive substances, which can cause a massive inflammatory response. More directed approaches that selectively target the cells before activation are needed.

### SUMMARY

In view of the shortcomings of the prior art, there is a need for new compounds that selectively target mast cells and eosinophils without causing their activation.

The present disclosure relies on the finding that certain populations of granulocytes can be selectively reduced by the use of monensin.

Monensin (Formula 1) is a polyether antibiotic isolated from Streptomyces Cinnamonensis and is widely used in ruminant animal feed additives and anti-coccidia drugs.

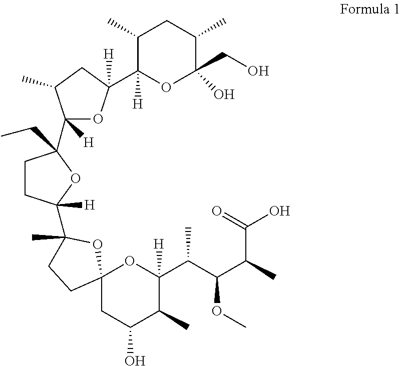 [SMILES: CC[C@]1([C@H]2O[C@H]([C@@H]3O[C@](O)(CO)[C@@H](C)C[C@H]3C)C[C@H]2C)CC[C@@H]([C@@]2(C)CC[C@@]3(C[C@@H](O)[C@H](C)[C@H]([C@H](C)[C@H](OC)[C@H](C)C(=O)O)O3)O2)O1]

“Monensin” is known as Monensin A sodium salt; Monensin sodium; Monensin sodium salt; NSC 343257; Sodium monensin; Elancoban®; Elancogran®, Coban®, Rumensin®, Coxidin® and, has the IUPAC Names: Stereoisomer of 2-[2-ethyloctahydro-3′methyl-5′[tetrahydro-6-hydroxy-6-(hydroxymethyl)]-3,5-dimethyl-2Hpyran-2-yl] [2,2′-bifuran′5′yl] ]-9-hydroxy-βmethoxy-a, γ,2,8,-tetramethyl-1,6-dioxaspiro[4.5]decan-7-butanoic acid. and: 4-[2-[5-ethyl-5-[5-[6-hydroxy-6-(hydroxymethyl)-3,5-dimethyl-oxan-2-yl]-3-methyl-oxolan-2-yl]oxolan-2-yl]-9-hydroxy-2,8- dimethyl-1,6-dioxaspiro[4.5]dec7-yl]-3-methoxy-2-methyl-pentanoic acid.

The inventors have surprisingly found that monensin is a highly potent compound for inducing selective apoptosis in granulocytes comprising acidic cytoplasmic granules. Eosinophils and mast cells are examples of such granulocytes.

Hence, it is a first object to provide monensin for use in the alleviation, prevention and/or treatment of a condition or a disease, wherein said condition or disease is selected from conditions and diseases mediated by granulocytes comprising acidic cytoplasmic granules, as defined in claim 1.

It is also the object of the present invention to provide methods for alleviation, prevention and/or treatment of a condition or a disease, wherein said condition or disease is mediated by granulocytes comprising acidic cytoplasmic granules, said method comprising administering monensin to a subject suffering, or suspected of suffering, from such a condition or disease.

In another aspect, the condition or disease involves activation of granulocytes comprising acidic cytoplasmic granules, i.e. degranulation.

Further, it is demonstrated that monensin effectively reduces the number of granulocytes comprising acidic cytoplasmic granules. It was found that monensin selectively induces cell death in said granulocytes. The cell death was further demonstrated to be apoptosis. The apoptosis may be caspase independent apoptosis.

Monensin selectively induces apoptosis in granulocytes comprising acidic cytoplasmic granules, and the effect is more pronounced in mature granulocytes.

In a further aspect, said granulocytes are mast cells and/or eosinophils.

In a further aspect, said granulocytes are mast cells. In yet another aspect, said granulocytes are eosinophils.

In a further aspect, said condition or disease may be an inflammatory condition or disease. The condition or disease may further be selected from bronchitis, bronchoconstriction, inflammatory skin diseases, allergic asthma, and anaphylaxis.

In a further aspect, said condition or disease is selected from mastocytosis, mast cell activation syndrome, a type I hypersensitivity, cardiovascular disorders, hereditary alpha-tryptasemia, and cancer.

In some aspects, the type I hypersensitivity is selected from allergic asthma, allergic rhinitis, allergic conjunctivitis, allergic dermatitis, anaphylaxis, urticaria, angioedema.

In a further aspect, the cardiovascular disorder is selected from atherosclerosis, cardiac fibrosis, and Kounis syndrome.

In a further aspect, said granulocytes are eosinophils.

In a further aspect, said condition or disease is selected from hypereosinophilic syndrome, eosinophilic gastrointestinal disorders, eosinophilic asthma, eosinophilic granuloma, eosinophilia-myalgia syndrome, Kimura disease, and angiolymphoid hyperplasia with eosinophilia. In a further aspect monensin is administered in a pharmaceutically effective dose to a subject in need thereof.

Monensin for use as described in the present disclosure wherein administration of monensin is performed by inhalation, oral, peroral, mucosal, or topical administration.

In yet another aspect, monensin may be used in combination with one or more additional anti-inflammatory therapy/therapies.

It is a further object to provide a pharmaceutical composition comprising the pharmacologically active compound monensin for use in the alleviation, prevention and/or treatment of a disease or condition, wherein said condition or disease is mediated by granulocytes comprising acidic cytoplasmic granules.

It is a further object to provide methods for alleviation, prevention and/or treatment of a disease or condition, wherein said condition or disease is mediated by granulocytes comprising acidic cytoplasmic granules, said method comprising administering a pharmaceutical composition comprising the pharmacologically active compound monensin to a subject suffering, or suspected of suffering, from such a condition or disease.

The pharmaceutical composition may also be used in the alleviation, prevention and/or treatment of a condition or a disease involving activation of granulocytes.

The pharmaceutical composition may further comprise a pharmaceutically acceptable carrier and/or excipient.

In yet another aspect, administration of the pharmaceutical composition is performed by intravenous injection, intramuscular injection, intraperitoneal injection, subcutaneous injection or infusion, thecal, peroral, mucosal, inhalation or topical administration. Preferably, administration of the pharmaceutical composition is performed by inhalation, oral, peroral, mucosal, or topical administration.

The formulation of the pharmaceutical composition of the present disclosure may be in the form of a tablet, a powder, a gel, a capsule, an ointment, an aerosol, or a lyophilizate.

In a further aspect the pharmaceutical composition comprising monensin is administered in a pharmaceutically effective dose to a subject in need thereof.

In yet a another aspect Monensin as discussed above or the pharmaceutical composition discussed above is administered in a pharmaceutically effective dose, to a subject in need thereof

In yet another aspect, the pharmaceutical composition may be used in combination with one or more additional anti-inflammatory therapy/therapies. It is a further object of the present invention to provide for the use of monensin in the manufacture of a pharmaceutical composition for use in a method of treatment according to the above.

In yet another aspect, the administration of monensin or the pharmaceutical composition comprising monensin is used in combination with one or more additional treatment(s). The other treatment may for example be an anti-inflammatory treatment/therapy, a treatment that inhibits activation of said cells, i.e degranulation, or block the action of allergic mediators or activators. The administration of these agents can take place simultaneously or at different times, or in combination.

Several medications may be used to block the action of allergic mediators, or to prevent activation of cells and degranulation processes, these include antihistamine drugs, acting by blocking histamine action on nerve endings, glucocorticoids and epinephrine (adrenaline). Cromoglicate-based drugs (sodium cromoglicate, nedocromil) block a calcium channel essential for mast cell degranulation, stabilizing the cell and preventing release of histamine and related mediators. Leukotriene antagonists (such as montelukast and zafirlukast) block the action of leukotriene mediators and are being used increasingly in allergic diseases. Anti-cholinergics, decongestants, and other compounds thought to impair eosinophil chemotaxis, are also commonly used. Therapies such as mast cell and/or eosinophil-targeted therapies.

Therapies against eosinophilic airway inflammation include monoclonal antibodies against IL-5 or IL-5 receptor alpha (mepolizumab, reslizumab, and benralizumab), IL-13 (lebrikizumab and tralokinumab), IL-4 receptor alpha (dupilumab), IgE (omalizumab), and anti-thymic stromal lymphopoietin (tezepelumab) and small molecule therapies such as prostaglandin D₂ blockers (fevipiprant and timapiprant).

In view of the present disclosure, a combination treatment in which monensin is administered in combination with one or more other anti-inflammatory drugs would provide a more efficient therapy to a subject in need thereof.

The present disclosure further provides a method for alleviating, preventing and/or treating a condition or a disease mediated by granulocytes. The granulocytes have acidic cytoplasmic granules, such as mast cells and eosinophils. The method comprising administering a pharmacologically effective dose of monensin to a subject in need of such treatment. The condition or disease may for example be an inflammatory condition or disease, such as bronchitis, bronchoconstriction, inflammatory skin diseases, allergic asthma, and anaphylaxis.

Further, the administration is performed by for example intravenous, intramuscular, intraperitoneal, subcutaneous injection or infusion, thecal, oral, peroral, mucosal, inhalation or topical administration.

Further, the pharmaceutical composition comprising monensin may be formulated in the form of a tablet, a powder, a gel, a capsule, an ointment, an aerosol, or a lyophilizate.

The method may also be performed in combination with one or more additional anti-inflammatory therapy/therapies.

The advantages described herein will be realized and attained by, at least in part, means of the elements and combinations particularly pointed out in the appended claims.

### BRIEF DESCRIPTION OF DRAWINGS

FIG. 1 Shows kinetic analyses of cytotoxic effects of the test compounds parthenolide, clomiphene, auranofin and monensin on bone marrow-derived mast cells (BMMC).

FIGS. 2A-F Show that Monensin selectively causes apoptotic cell death in mast cells (MCs).

FIG. 3 Shows that monensin induces caspase-independent cell death in mast cells.

FIGS. 4A-G Show that mast cell death induced by monensin is dependent on acidic mast cell secretory granules in BMMCs.

FIGS. 5A-C Show that monensin does not cause mast cell activation.

FIGS. 6A-E Show that mast cells (B) and eosinophils (E) are sensitive to monensin whereas lymphocytes (C) and macrophages (D) are refractory.

FIGS. 7A-E Show that monensin selectively induce cell death on human peripheral blood-derived cell populations, gating (A), eosinophils (B), monocytes (C), lymphocytes (D) and neutrophils (E).

FIGS. 8A-D Show that monensin abrogates airway reactivity in trachea isolated from house dust mite (HDM)-sensitized guinea pigs (A), Carbacol (B), mast cells with intact nucleus (C), tracheal tissues from HDM-sensitized guinea pigs were challenged with HDM extract, followed by measurement of bronchoconstriction (D).

### TERMS AND ABBREVIATIONS

All terms as used herein are intended to have the meaning commonly given to them in the relevant art. For the sake of clarity, a few terms are specifically discussed below.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

As used herein, the term “dose amount” refers to the quantity, e.g., milligrams (mg), of the substance which is administered to the subject. In one embodiment, the dose amount is a fixed dose, e.g., is not dependent on the weight of the subject to which the substance is administered. In another embodiment, the dose amount is not a fixed dose, e.g., is dependent on the weight of the subject to which the substance is administered, or for a topical therapy a dose may be related to the surface area that is treated, e.g. dose/m² of skin.

As used herein, the term “periodicity” as it relates to the administration of a substance refers to a (regular) recurring cycle of administering the substance to a subject.

The “duration of a periodicity” refers to a time over which the recurring cycle of administration occurs.

As used herein, and unless otherwise specified, the terms “treat,” “treating” and “treatment” refer to an action that occurs while a patient is suffering from a disease, which reduces the severity of a disease, or retards or slows the progression of the disease, or achieving or maintaining a therapeutic objective. An “effective patient response” refers to any increase in the therapeutic benefit to the patient.

The phrase “pharmaceutically acceptable” are employed herein to refer to those compounds, materials, compositions, and/or dosage forms which are, within the scope of sound medical judgment, suitable for use in contact with the tissues of the subject with toxicity, irritation, allergic response, or other problems or complications, commensurate with a reasonable benefit/risk ratio. Compounds, materials, compositions, and/or dosage forms that are pharmaceutically acceptable are also considered cosmetically acceptable.

The phrase “pharmaceutically acceptable excipient” as used herein refers to an acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the, optionally therapeutic, compound for administration to the subject. Each excipient should be “acceptable” in the sense of being compatible with the other ingredients of the formulation and not injurious to the subject. Some examples of materials which can serve as pharmaceutically excipients include: ethanol, sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations. If desired, certain sweetening and/or flavoring and/or coloring agents may be added. Pharmaceutically acceptable excipients are also considered cosmetically acceptable excipients.

### DETAILED DESCRIPTION

Different aspects of the present disclosure will be described more fully hereinafter by means of examples and with reference to the enclosed drawings. The embodiments disclosed herein can, however, be realized in many different forms and should not be construed as being limited to the aspects set forth herein.

It is to be understood that the configurations, process steps, and materials disclosed herein may vary somewhat within the scope of the appended claims.

As set out herein, mast cell activation and eosinophil activation can result in the release of an impressive array of potent bioactive substances (degranulation), which can cause a massive inflammatory response. Mast cells and eosinophils are known to have an aggravating impact on a range of debilitating human diseases and strategies to dampen their harmful activities are therefore highly demanded. The present invention provides monensin as an active pharmaceutical agent to achieve this goal.

Anti-mast cell and anti-eosinophil drugs or agents are drugs or agents which dampen the harmful activities of mast cells and/or eosinophils. Such drugs are said to exhibit mast cell inhibiting properties or anti-mast cell properties and eosinophil inhibiting properties or anti-eosinophil properties, respectively. These properties include, in this context, cytotoxicity, inhibition of mast cell/eosinophil activation, induction of reactive oxygen species (ROS), and induction of apoptosis/necrosis in mast cells/eosinophils.

Several medications may be used to block the action of allergic mediators, or to prevent activation of cells and degranulation processes, these include antihistamine drugs which acts by blocking histamine action on nerve endings, glucocorticoids and epinephrine (adrenaline).

Cromoglicate-based drugs (sodium cromoglicate, nedocromil) block a calcium channel essential for mast cell degranulation, stabilizing the cell and preventing release of histamine and related mediators. Leukotriene antagonists (such as montelukast and zafirlukast) block the action of leukotriene mediators and are being used increasingly in allergic diseases. Anti-cholinergics, decongestants, and other compounds thought to impair eosinophil chemotaxis, are also commonly used. Although rare, the severity of anaphylaxis often requires epinephrine injection.

In view of the present disclosure, a combination treatment in which monensin is administered in combination with one or more other anti-mast cell drugs may provide a more efficient therapy to a subject in need thereof.

In some aspects, the present invention relates to alleviation, prevention and/or treatment of a condition or disease, wherein said condition or disease is mediated by mast cells.

Mast cells are known to mediate a number of diseases, including mastocytosis, mast cell activation syndrome, type I hypersensitivity, cardiovascular disorders, hereditary alpha-tryptasemia, and cancer.

Mastocytosis is a rare neoplastic disorder characterized by a clonal proliferation of mast cells and accompanied by aberrant mast cell activation. The abnormal increase of mast cells may occur in only the skin (cutaneous mastocytosis), in extracutaneous tissues involving multiple organs (systemic mastocytosis), or in solid tumors (mastocytoma). The most common form of cutaneous mastocytosis is known as urticaria pigmentosa and occurs primarily in children. Mastocytosis also includes mast-cell sarcoma.

Type I hypersensitivity involves mast cell degranulation and release of inflammatory mediators such as histamine. Type I hypersensitivities include atopic and allergic diseases, including allergic asthma, allergic rhinitis, allergic conjunctivitis, allergic dermatitis, anaphylaxis, urticarial, angioedema,

Mast cells have also been shown to mediate a number of cardiovascular disorders such as atherosclerosis, cardiac fibrosis, and Kounis syndrome (Hermans et al. Int. J. Mol. Sci. 2019, 20(14), 3395).

Activated mast cells also contribute to cancer pathologies as they can release many pro-angiogenic and tumor growth stimulatory mediators. It has therefore been suggested that modulating i.a. mast cell viability at malignant sites can be of importance to control tumor growth (Kormelink et al. Current Pharmaceutical Design, Volume 15, Number 16, 2009, pp. 1868-1878(11)).

In some aspects, the present invention relates to alleviation, prevention and/or treatment of a condition or disease, wherein said condition or disease is mediated by eosinophils. The term “eosinophil-associated diseases” (EAD) refers to a spectrum of disorders in which eosinophils are believed to play a central role in the pathophysiology, i.e. mediating the condition or disease. Some disorders, including hypereosinophilic syndrome (HES) and eosinophilic gastrointestinal disorders (EGID) are EAD by definition; other (more common) diagnoses have clinical subtypes that are considered EAD. Among these, the best example is eosinophilic asthma, a subtype of asthma characterized by sputum eosinophilia≥2% and peripheral eosinophilia. Further diseases mediated by eosinophils include eosinophilic granuloma, eosinophilia-myalgia syndrome, Kimura disease, and angiolymphoid hyperplasia with eosinophilia.

Whereas topical and/or systemic corticosteroids remain the cornerstone of therapy for EAD and are initially effective in controlling eosinophilia and symptoms in many patients, resistance and toxicity become increasingly common over time and second-line agents are often needed. Anti-eosinophil drugs or agents are drugs or agents which dampen the harmful activities of the eosinophils, these include monoclonal antibodies against IL-5 or IL-5 receptor alpha (mepolizumab, reslizumab, and benralizumab), IL-13 (lebrikizumab and tralokinumab), IL-4 receptor alpha (dupilumab), IgE (omalizumab), and anti-thymic stromal lymphopoietin (tezepelumab) and small molecule therapies such as prostaglandin D₂ blockers (fevipiprant and timapiprant).

In view of the present disclosure, a combination treatment in which monensin is administered in combination with one or more other anti-eosinophil cell drugs may provide a more efficient therapy to a subject in need thereof.

In this specification, unless otherwise stated, the term “pharmaceutically active compound” encompasses any substance that will produce a therapeutically beneficial pharmacological response when administered to a host, including both humans and animals.

In this specification, unless otherwise stated, the term “pharmaceutically acceptable excipient” means a non-toxic, inert solid, semi-solid or liquid filler, diluent, encapsulating material or formulation auxiliary of any type.

In this specification the term “administering”, or “administration” means providing a drug to a subject in a manner that is pharmacologically useful.

The expressions “anti-mast cell drug”, “anti-mast cell agent” and “anti-mast compound” and the like are used interchangeably and are meant to include drugs and agents that dampens the harmful activities of mast cells.

Apoptosis is a form of programmed cell death that occurs in multicellular organisms and involves biochemical events leading to characteristic cell changes (morphology) and death. These changes include blebbing, cell shrinkage, nuclear fragmentation, chromatin condensation, chromosomal DNA fragmentation, and global[vague] mRNA decay. In contrast to necrosis, which is a form of traumatic cell death that results from acute cellular injury, apoptosis is a highly regulated and controlled process that confers advantages during an organism's life cycle. Necrosis is caused by factors external to the cell or tissue, such as infection, or trauma which result in the unregulated digestion of cell components.

Caspase-independent cell death (CICD) is cell death which ensues when a signal that normally induces apoptosis fails to activate caspases. Even so, CICD often shares common characteristics with apoptotic cell death. These include upstream signaling pathways that are critical for both forms of cell death such as mitochondrial outer membrane permeabilization (MOMP). Archetypal caspase-dependent events such as phosphatidylserine externalization and wide-scale chromatin condensation are notably absent during CICD. Cells undergoing CICD often display large-scale cytoplasmic vacuolization, autophagosome accumulation and peripheral nuclear condensation. In addition, CICD generally proceeds with much slower kinetics than apoptosis. Furthermore, although apoptotic cells exhibit a relatively invariant phenotype, cells undergoing CICD may display widely varying characteristics dependent upon factors such as the initial stimulus, cell type and so on.

Decorti et al. reported in Agents and Actions, vol. 32, 3/4 (1991) that monensin (0.6-150 μg/ml) was able to prevent histamine release from mast cells stimulated by various secretagogues such as the calcium ionophore A23187. However, when incubated for longer periods (5 hr) with mast cells, monensin was shown to be a potent secretagogue, and induced an extremely slow and progressive histamine release, which, at least for lower doses, was noncytotoxic. Monensin stimulated histamine secretion was strictly dependent on extracellular Na⁺ concentrations, and independent on extracellular Ca²⁺ concentrations. It is to be noted that Decorti et al. used monensin in a concentration of 50 μg/mL, which corresponds to ˜72 μM.

According to the present disclosure, it was found that nanomolar concentrations of monensin, i.e. much lower concentrations than used by Decorti, can be used to selectively induce apoptosis in mast cells and eosinophils. This cytotoxic effect on mast cells and eosinophils at nanomolar concentrations exhibited by monensin highlights its potential as a potent and selective compound for developing a successful and useful anti-inflammatory therapy.

The medicaments, pharmaceutical compositions or therapeutic combinations according to the present invention may be in any form suitable for the application to humans and/or animals, preferably humans including infants, children and adults and can be produced by standard procedures known to those skilled in the art. The medicament, (pharmaceutical) composition or therapeutic combination can be produced by standard procedures known to those skilled in the art, e.g. from “Pharmaceutics: The Science of Dosage Forms”, Second Edition, Aulton, M. E. (ED. Churchill Livingstone, Edinburgh (2002); “Encyclopedia of Pharmaceutical Technology”, Second Edition, Swarbrick, J. and Boylan J. C. (Eds.), Marcel Dekker, Inc. New York (2002); “Modern Pharmaceutics”, Fourth Edition, Banker G. S. and Rhodes C. T. (Eds.) Marcel Dekker, Inc. New York 2002 y “The Theory and Practice of Industrial Pharmacy”, Lachman L., Lieberman H. And Kanig J. (Eds.), Lea & Febiger, Philadelphia (1986). The respective descriptions are hereby incorporated by reference and form part of the disclosure.

An effective dose of monensin may include a “therapeutically effective dose or amount” or a “prophylactically effective dose or amount” as defined above. A therapeutically effective amount may vary according to factors such as the disease state, age, sex, and weight of the individual, and the ability to elicit a desired response in the individual. A therapeutically effective dose/amount is also one in which any toxic or detrimental effects are outweighed by the therapeutically beneficial effects. A “prophylactically effective dose/amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single dose may be administered, several divided doses may be administered over time or the dose may be proportionally reduced or increased as indicated by the exigencies of the therapeutic situation. The dose may be administered to the subject upon symptoms, or before onset of symptoms.

It is to be noted that dosage values may vary with the type and severity of the condition to be alleviated, prevented or treated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### Materials and Methods

### Cells

Bone marrow-derived mast cells (BMMCs), peritoneal cell-derived mast cells (PCMCs), human skin mast cells (hskinMC), HEK-293, immature human mast cell lines (LUVA and HMC-1) cells were cultured as previously described [Alanazi, S.; Melo, F. R.; Pejler, G. Histone Methyltransferase Inhibition Has a Cytotoxic Impact on Transformed Mast Cells: Implications for Mastocytosis. Anticancer Res. 2020, 40, 2525-2536]. Primary human small airway epithelial cells and human fetal lung fibroblasts were also assessed.

### Screening

10 mM stock solutions of the compounds auranofin, clomiphene, monensin and parthenolide (see Table 1) in DMSO were spotted at 100 nL in 96-microtiter plates. BMMCs (50,000) were added in 100 μL culture media (final compound concentration of 10 μM) followed by incubation at 37° C. for 24 hours. Cell viability was assessed by using Presto Blue™.

### FACS Staining of Mouse and Human Cells

Mouse peritoneal cells were treated with monensin and stained with labeled antibodies against CD117 (2B8), FcεRI (MAR-1), Siglec-F (E50-2440), CD11b (M1/70), F4/80 (BM8), CD3 (17A2), CD4 (GK1.5), CD8b (eBioH35-17.2) and CD19 (ebuo1D3).

Mononuclear cells and granulocytes were purified from heparinized human peripheral blood. Cells were stained with antibodies against CD4 (RPA-T4), CD8 (RPA-T8), CD19 (HIB19) and CD14 (M5E2) or CCR3 (5E8) and CD15 (W6D3) for analysis of mononuclear cells and granulocytes, respectively.

### Assessment of Cell Death, pH in Acidic Organelles, Intracellular pH, and Intracellular Calcium

To assess cell death, cells were stained with Annexin V and either DRAQ7™ or Via-Probe™ Red Nucleic Acid Stain and analyzed by flow cytometry. To probe pH in acidic organelles, cells were incubated with LysoSensor Blue DND-167, followed by flow cytometry. The relative intracellular pH changes were assessed using the cell-permeable pH-sensitive fluorescent indicator (BCFL-AM). Intracellular calcium changes were assessed using Fluo-4/AM.

### Measurement of Antigen-Induced Bronchoconstriction Ex Vivo

Smooth muscle contractions of tracheal segments from house dust mite (HDM) sensitized guinea pigs were measured in the organ bath system (EMKA, Paris, France) after culturing with monensin.

### Statistical Analysis

Statistical differences between groups were determined using one-way ANOVA with post hoc Tukey's multiple comparison test unless otherwise stated. p-values of less than 0.05 were considered significant (*p<0.05; ** p<0.01; *** p<0.001; *** p<0.0001). Experiments were performed in triplicates or quadruplicates.

### Results

### Identification of Drugs with Cytotoxic Activity Against Mast Cells

In a search for novel agents that selectively reduce the viability of mast cells, a library of drug compounds was screened, and a number of compounds were found to have a cytotoxic effect on BMMCs. The viability of the cells after treatment with the compounds was measured using reduction of resazurin to a fluorescent product as readout. A number of candidate drugs were selected for further evaluation. Out of the candidate drugs, an initial evaluation of their potential as clinical anti-mast cell agents was made. Compounds with known histamine receptor 1 (H1) antagonist, anti-inflammatory, and antibacterial properties, as well as drugs used for endocrine and metabolic indications were evaluated. As an additional readout for cytotoxic activity, cells treated for trypan blue positivity were assessed. These analyses showed that most of the compounds affecting viability also produced cell death as judged by Trypan blue positivity (data not shown).

### Candidate Anti-Mast Cell Compounds

Four drugs were tested: auranofin, clomiphene, monensin and parthenolide at 1 & 10 μM and incubated with mast cells (see under material & methods) whereafter the viability of the mast cells was evaluated. The result is shown in Table 1.

TABLE 1
Test of different drugs

| | | | | | |
|---|---|---|---|---|---|
| | | | | | Caspase |
| | | Viability | Viability | Viability | 3 & 7 |
| Anti-MC | | % | % | % (TB) | acti- |
| drugs | Family | (1 μM) | (10 μM) | (10 μM) | vation |

Anti-inflammatory:

| | | | | | |
|---|---|---|---|---|---|
| Parthenolide | Anti-inflam- | 102 | 59 | 11 | + |
| | matory | | | | |

Antiseptic/antibacterial:

| | | | | | |
|---|---|---|---|---|---|
| Monensin | Anti- | 72 | 66 | 12 | − |
| | bacterial | | | | |

Metabolism:

| | | | | | |
|---|---|---|---|---|---|
| Auranofin | Anti- | 83 | 1 | 100 | + |
| | rheumatic | | | | |

Endocrinology:

| | | | | | |
|---|---|---|---|---|---|
| Clomiphene | Anti- | 99 | 76 | 22 | − |
| | infertility | | | | |

Moreover, caspase-3 & 7 positivity was seen in response to two of the compounds, parthenolide and auranofin, indicating apoptotic cell death. Notably, monensin and clomiphene were found to induce cell death in the absence of a positive signal for caspase-3 & 7 activation (Table 1).

To further evaluate the anti-MC properties of these compounds, experiments to assess their efficacy over time and at different concentrations were performed (Example 1).

### Example 1 Auranofin, Clomiphene, Monensin and Parthenolide are Cytotoxic to Mast Cells

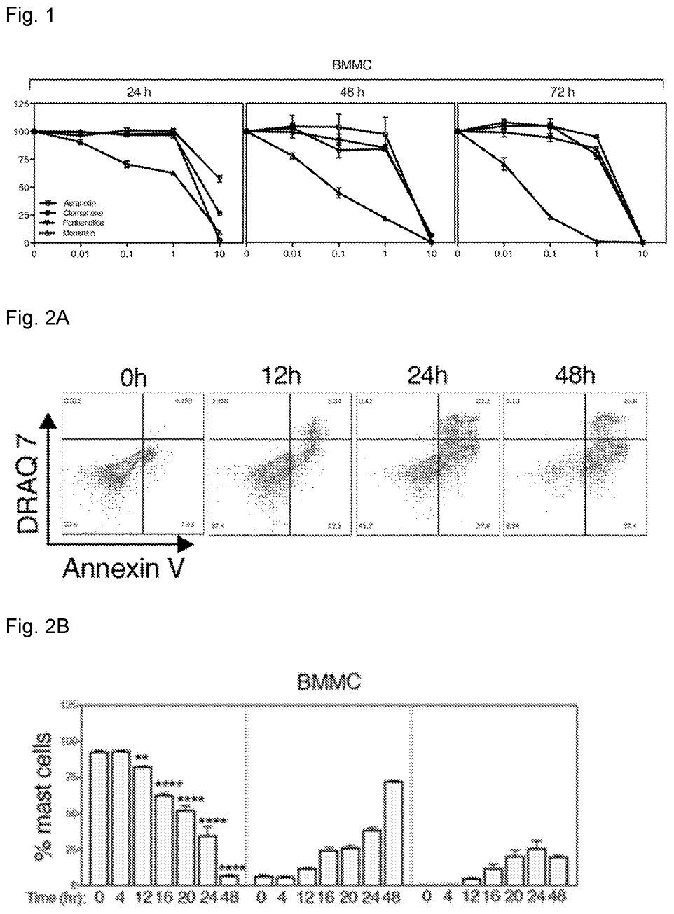

Mast cells (BMMCs; A; 50×10³ cells) were incubated with: auranofin, clomiphene, monensin and parthenolide at 0.01-10 μM for 24, 48 or 72 h (FIG. 1).

Monensin was found to be markedly more potent than any of the other three drugs, showing considerable toxicity at concentrations down to 10 nM, especially after prolonged (72 h) incubation times.

Due to the high cytotoxic activity of monensin to mast cells, the cytotoxicity against other cell types was also tested. Five thousand human primary airway epithelial cells (HSAEC), HEK293 cells or human fibroblast cells (HFL-1) were incubated with auranofin, clomiphene, monensin or parthenolide at 0.01-10 μM for 48 h. Only monensin exhibited a cytotoxic effect on the cells from 1 μM, even if it was much less compared to the cytotoxic effect on mast cells, all drugs exhibited a cytotoxic effect at 10 μM on HSAEC and HEK293 cells, whereas only monensin and auranofin exhibited cytotoxicity on HSFib (HFL-1) cells (data not shown). Residual cell viability was measured using PrestoBlue™.

In conclusion, monensin was shown to be more cytotoxic to mast cells than to either the primary human fibroblast cell line HFL-1, primary human small airways epithelial HSAEC cells or the human cancer epithelial HEK-293 cell line. These results clearly show that monensin exhibit a high and selective cytotoxic effect on mast cells.

### Example 2. Monensin Induces Apoptotic Cell Death in Different Mast Cell Populations

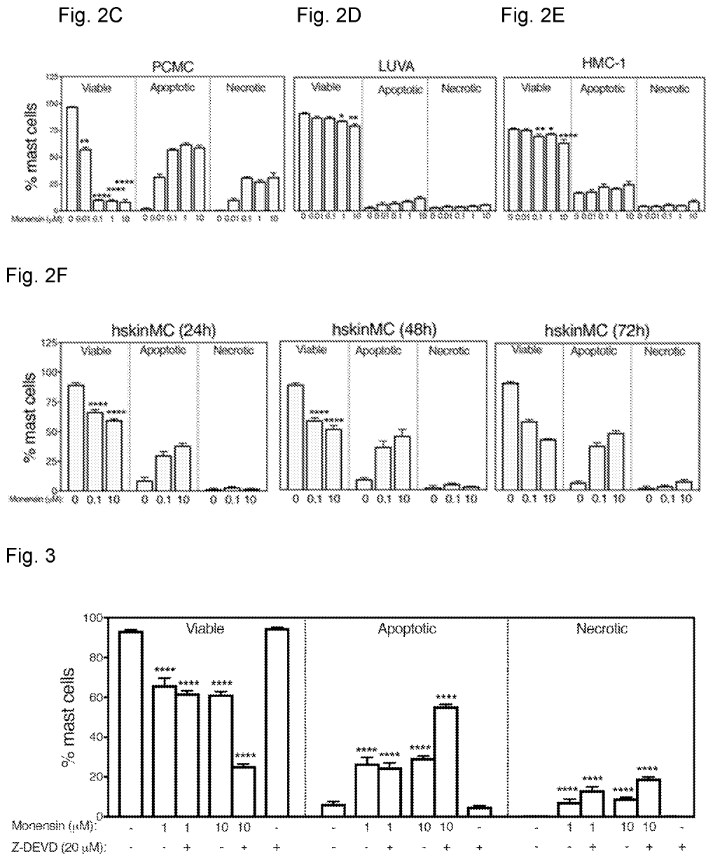

To analyze the mechanism by which monensin acts on mast cells, Annexin V/DRAQ7 staining was first adopted to assess the type of cell death induced by monensin, the gating is shown in FIG. 2A. The analysis revealed that monensin induced predominantly Annexin V single positivity (Annexin V*/DRAQ7⁻) in mouse bone-marrow-derived mast cells (BMMCs), which is a sign of apoptotic cell death middle panel (FIG. 2B). Necrotic/late apoptotic (Annexin V⁺/DRAQ7⁺) cells were also seen, but to a lesser extent (right panel).

BMMCs are commonly used to study mast cell function and have properties that largely resemble those of mast cell populations found in vivo. However, they are somewhat less mature in terms of granular content than the connective tissue type mast cells found in, e.g. skin and peritoneum.

To assess whether monensin also induces cell death of fully mature mast cells, mouse peritoneal cell-derived mast cells (PCMCs) were developed and their sensitivity to monensin was assessed. As seen in FIG. 2C, these experiments revealed that monensin was highly cytotoxic to the PCMCs too. In fact, these mature mast cells were even more sensitive to monensin than were the BMMCs, with significant cell death seen at concentrations down to 10 nM (24 h incubation).

Next, the inventors assessed if monensin is cytotoxic for human mast cells by assessing its effect on immature human mast cell lines (LUVA, HMC-1) and on fully mature skin mast cells (hskinMC). These analyses showed that monensin was cytotoxic to the LUVA (FIG. 2D) and HMC-1 cells (FIG. 2E), albeit with a lower potency than for BMMCs and PCMCs. In contrast, human skin mast cells (hskinMC) showed considerable sensitivity to monensin especially after longer incubation time (right panel), monensin caused mainly apoptosis in the hskin cells, the sign of necrosis was almost negligible (FIG. 2F).

Altogether, these data suggest that fully mature mast cells are highly sensitive to monensin, whereas less mature mast cell populations were found to be less sensitive.

### Example 3 Monensin Induces Caspase-Independent Cell Death in Mast Cells

MCs were treated with 1 or 10 μM monensin, either in the absence or presence of a caspase inhibitor: Z-DEVD. Flow cytometry was used to distinguish between viable (Annexin V⁻/DRAQ7⁻), apoptotic (Annexin V⁺/DRAQ7⁻) and necrotic (Annexin V⁺/DRAQ7⁺) cells. The results show that monensin does not cause caspase-3 & 7 activations, indicating a caspase-independent apoptosis. In line with the latter, the extent of cell death in response to monensin was not affected by the caspase inhibitor, Z-DEVD (FIG. 3).

### Example 4. Monensin Acts on Mast Cells via a Granule-Dependent Mechanism

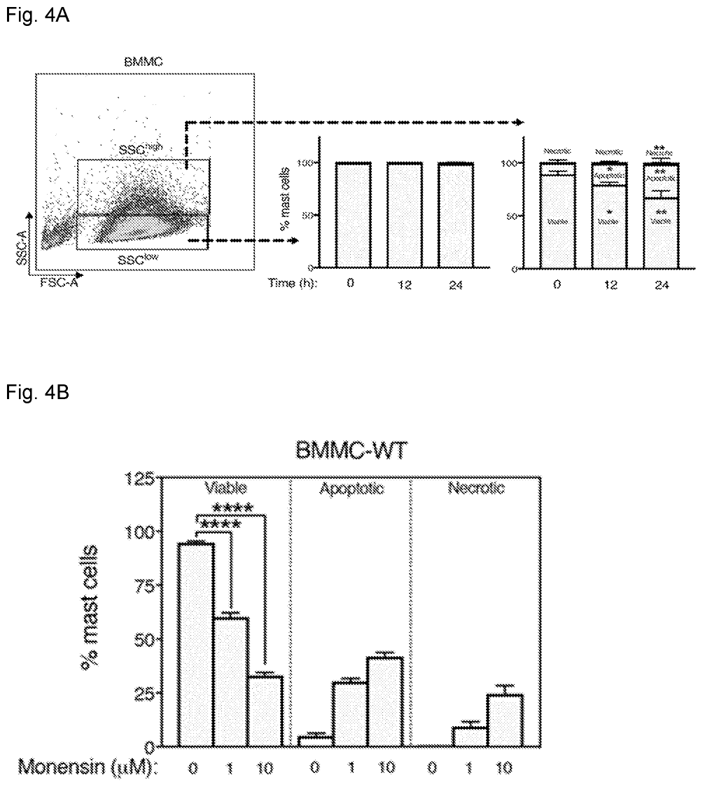

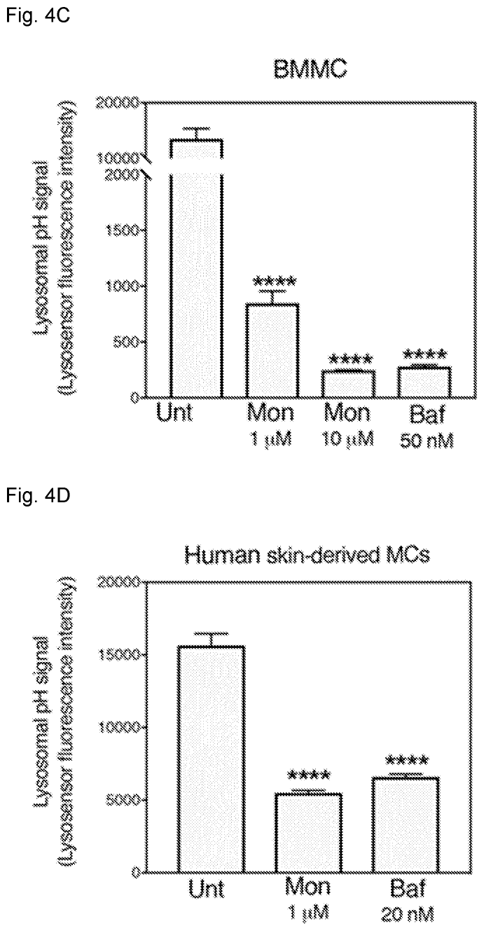

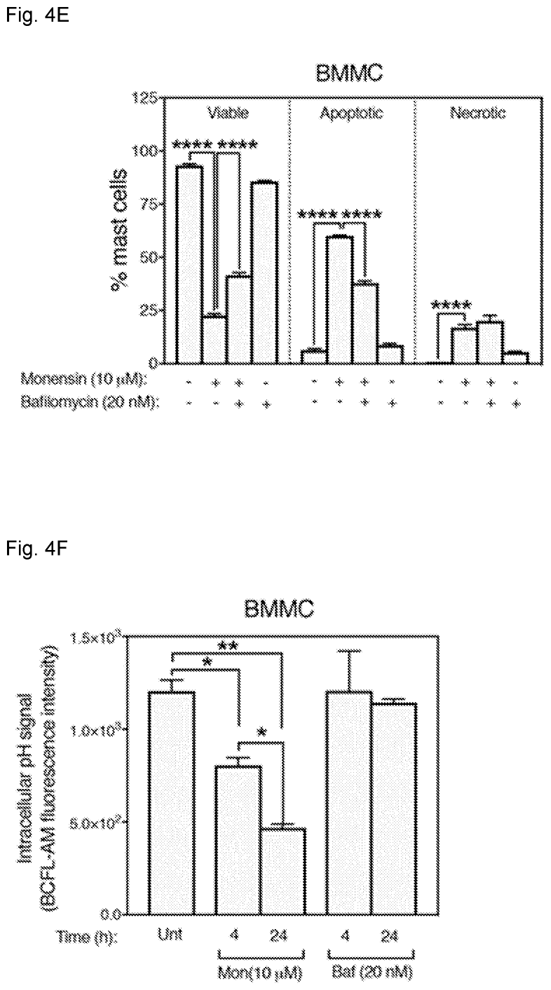

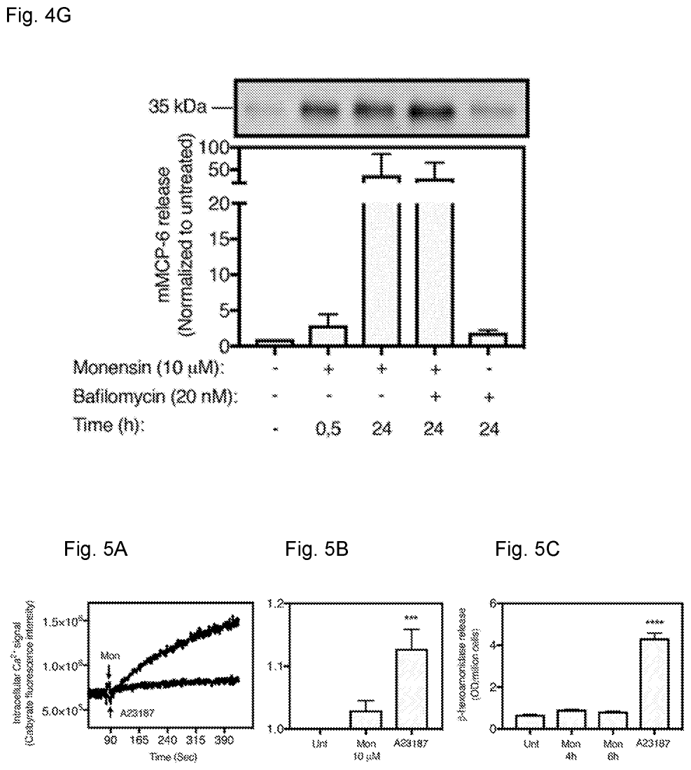

It has previously been shown that mast cells are remarkably sensitive to cell death by mechanisms targeting their secretory granules [Melo, F. R.; Waern, I.; Ronnberg, E.; Abrink, M.; Lee, D. M.; Schlenner, S. M.; Feyerabend, T. B.; Rodewald, H. R.; Turk, B.; Wernersson, S.; Pejler, G. A role for serglycin proteoglycan in mast cell apoptosis induced by a secretory granule-mediated pathway. J Biol Chem. 2011, 286, 5423-5433]. To provide further insight into this, mast cell populations were gated (FIG. 4A) for high and low maturity, as judged by side scatter intensity as a sign of granularity, and these subpopulations were assessed for sensitivity to monensin.

BMMCs were treated with 1 μM monensin for 12 and 24 h, followed by Annexin V/DRAQ7 staining to measure cell death. MCs were gated into cells having high and low maturity, as assessed by SSC analysis for granularity (A). The right panels depict the quantification of apoptotic (Annexin V⁺/DRAQ7⁻) and necrotic (Annexin V⁺/DRAQ7⁺) cell death in the respective populations (n=3). (B) BMMCs (0.5×10⁶ cells) were developed from WT (FIG. 4B) or serglycin^{-/-} (SG^{-/-}) mice (data not shown) and were treated with monensin 0-10 μM for 24 h, followed by cell death assessment (Annexin V/DRAQ7 staining). BMMCs (C; 0.5×10⁶ cells) or human skin MCs (0.1×10⁶ cells; D) were treated with monensin or bafilomycin A 0-10 μM for 30 min, followed by staining with LysoSensor Blue DND-167 (a lysosome probe sensitive to decreased pH) (n=4). BMMCs (0.5×10⁶ cells) were treated with either monensin or bafilomycin A (20 nM) alone or in combination (E), followed by measurement of cell death (Annexin V/DRAQ7 staining). (F) BMMCs were treated with monensin or bafilomycin A for 4 and 24 h, followed by assessment of cytosolic pH (n=2). This analysis showed that mast cells with high granularity (sign of maturation) were markedly more sensitive to monensin than were mast cells of low granularity (FIG. 4A). To further assess the impact of the secretory granules on monensin-induced cell death, it was evaluated whether the absence of serglycin, a granule-restricted proteoglycan, affects the response to monensin. Both WT and serglycin^{-/-} BMMCs were sensitive to cell death induced by monensin. However, whereas WT BMMCs underwent predominantly apoptotic cell death (confirming results above), serglycin^{-/-} BMMCs were predominantly Annexin V/DRAQ7 double-positive, suggesting death by necrosis (not shown).

These findings further support that the mast cell granules have a major impact on the cell death induced by monensin.

Next, it was tested whether monensin affects the acidity of the MC granules, as a sign of perturbed granule integrity. Indeed, monensin induced a marked reduction in the LysoSensor Blue DND-167 (lysosomal acidic pH probe) staining of both BMMCs and human skin MCs, suggesting that monensin causes granule permeabilization leading to elevation of granule pH (FIGS. 4C-D). To assess whether granule acidity is a prerequisite for monensin to induce cell death in mast cells, it was assessed whether blockade of granule acidification had an impact on the ability of monensin to induce mast cell apoptosis. For this purpose bafilomycin A was used to inhibit V-ATP:ase, the proton pump responsible for acidification of the mast cell granules. Bafilomycin A induced a profound reduction in granule acidity, confirming effective blockade of V-ATP:ase activity. Further, bafilomycin A caused a significant reduction in the extent of cell death induced by monensin (FIG. 4E). Together, these data show that the cytotoxic activity of monensin on MCs is dependent on the acidic pH of the mast cell secretory granules.

Conceivably, granule permeabilization would lead to leakage of protons into the cytosol, hence lowering the cytosolic pH. In support of this, treatment of MCs with monensin caused a significant drop in the cytosolic pH (FIG. 4F).

As further evidence for a mechanism involving granule permeabilization, monensin caused a profound translocation of mMCP-6, a major granule-contained protease, into the cytosolic fraction (FIG. 4G). The translocation of mMCP-6 to the cytosol was abrogated when cells had been treated with bafilomycin A, hence reinforcing that the effects of monensin on MCs is dependent on granule acidity.

### Example 5 Monensin Does Not Cause Mast Cell Activation

FIG. 5A and B shows how the intracellular Ca-levels (y-axis) in MCs are influenced by addition of monensin (lower curve) or A23187 (upper curve). Treatment of MCs with monensin did not cause an increase in cytosolic calcium, or release of hexosaminidase (C) suggesting that monensin does not cause MC activation.

### Example. 6 Ex Vivo-Derived MCs and Eosinophils are Sensitive to Monensin Whereas Macrophages and Lymphocytes are Refractory

To this end, monensin was added to cells recovered by peritoneal lavage of mice, followed by flow cytometry analysis to assess the effects of monensin on the various cell populations of the peritoneum.

Peritoneal cell populations were recovered by peritoneal lavage of mice. The gating strategy to identify viable cells and to identify macrophages, lymphocytes, MCs and eosinophils within the peritoneal cell population is shown in (A). Peritoneal cells were subjected to monensin at the indicated concentrations (0-10 μM) for 24 h, followed by cell death assessment by flow cytometry (Annexin V/Via-Probe staining). The panels show quantification of viable (Annexin V⁻/Via-Probe⁻) cells; in the MC (B), lymphocyte (C), macrophage (D) and eosinophil (E) populations (n=3).

These experiments confirmed that MCs were highly sensitive to monensin, being extensively depleted at monensin concentrations down to 10 nM (A-B). In contrast, macrophages and lymphocytes were resistant to monensin (C-D). It was also notable that eosinophils showed similar sensitivity to monensin as MCs, with substantial cell death seen at monensin concentrations down to 10 nM (E).

### Example. 7 Ex Vivo-Derived Human MCs and Eosinophils are Sensitive to Monensin Whereas Macrophages and Lymphocytes are Refractory

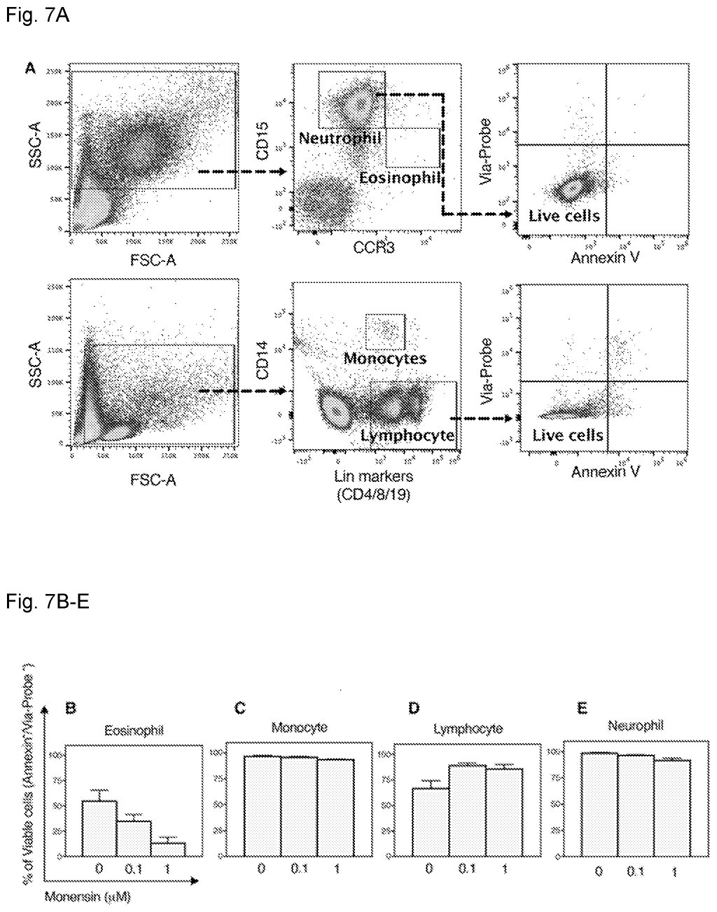

To extend these findings into the corresponding human leukocyte populations, the inventors assessed the effects of monensin on eosinophils (B), human blood monocytes (C), lymphocytes (D), and neutrophils (E). As seen in FIGS. 7A-C, similar to the mouse, human eosinophils were sensitive to monensin, whereas no reduction in the viability of either human monocytes or lymphocytes in response to monensin was seen. Further, human neutrophils were only marginally affected by monensin (D).

### Example 8 Monensin Abrogates Airway Reactivity in Trachea Isolated from House Dust Mite (HDM)-Sensitized Guinea Pigs

In order to evaluate if monensin could have an impact on a pathophysiological response where mast cells are implicated, the effect on allergic responses in airways with naturally occurring tissue-resident mast cells were investigated.

Tracheal tissues from HDM-sensitized guinea pigs were challenged with HDM extract, followed by measurement of bronchoconstriction (A). Tracheal tissue was then incubated with monensin at either 1 or 10 μM for 24-72 h. Bronchoconstriction was assessed (“force”; A) (“mN”, B). To assess tissue integrity, the isolated trachea was treated with carbachol (B), followed by measurements of bronchoconstriction (A).

Tracheal segments from guinea pigs, two weeks after sensitized with house dust mite (HDM), were excised, and cultured for 48 hours, in absence and presence of monensin (1 or 10 μM; D). In tissue baths, HDM induced a strong contraction, which at the highest concentration (1 μg/mL) reached almost 60% of maximal contraction induced by carbachol (A). This HDM-induced contraction was almost completely abolished in the segments cultured with both concentrations of monensin. At 1 μM, monensin did not affect the carbachol-induced contraction, whereas 10 μM of monensin decreased the carbachol concentration response curve (B).

Double staining of control (D) and monensin-treated tracheal tissue (E, F) was performed with astra blue.

Histological analysis of the tracheal segments showed that monensin caused a profound reduction in the proportion of mast cells having a defined nucleus (E). The latter findings suggest that monensin causes apoptosis of mast cells populating the tracheal tissue.

Taken together, the results shown here indicate that monensin have an impact on a pathological response where mast cells are involved.

Conceptually, the present disclosure provides a compound and method for selective depletion of granulocytes having an acidic granula, such as mast cell and eosinophil populations which constitute a highly efficient regime to accomplish this.

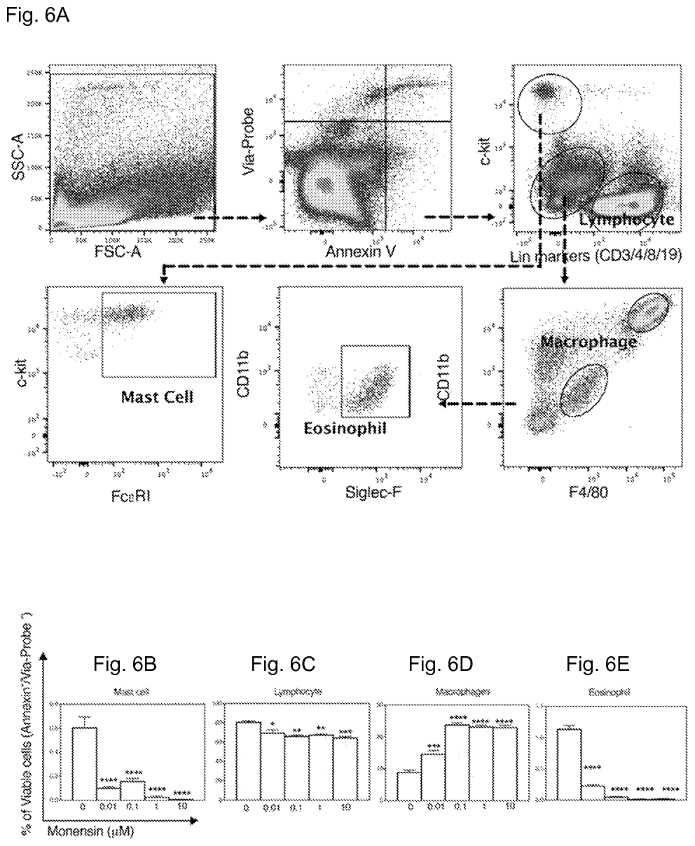

Considering the detrimental impact of mast cells in a wide range of human diseases, there is currently a demand for novel medical strategies to limit their actions. Here the inventors identified monensin as a novel, highly selective anti-mast cell compound (FIG. 6B). Further, monensin showed the same selective cytotoxicity to eosinophils (FIG. 6E).

Mast cells of low granularity (maturity) being markedly less sensitive to monensin than mast cells with a high granule content (mature) (FIG. 4).

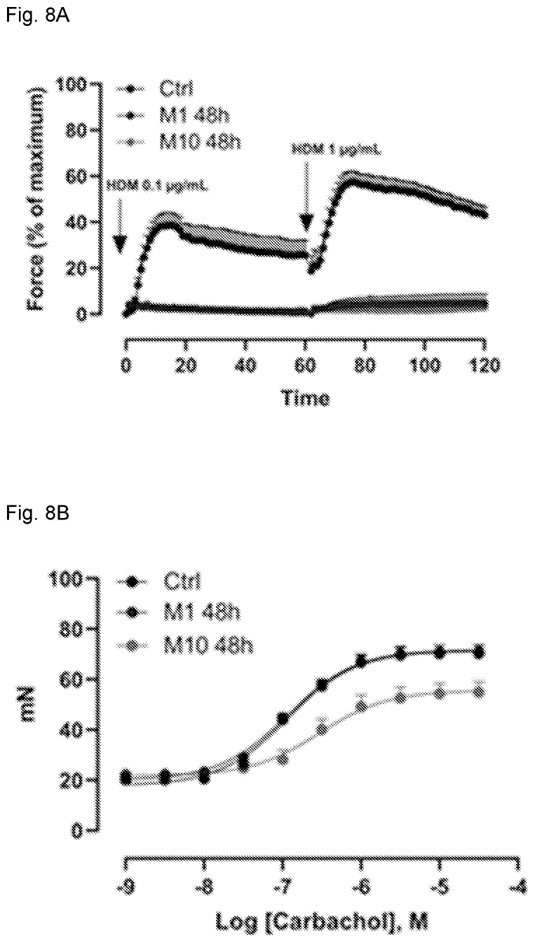

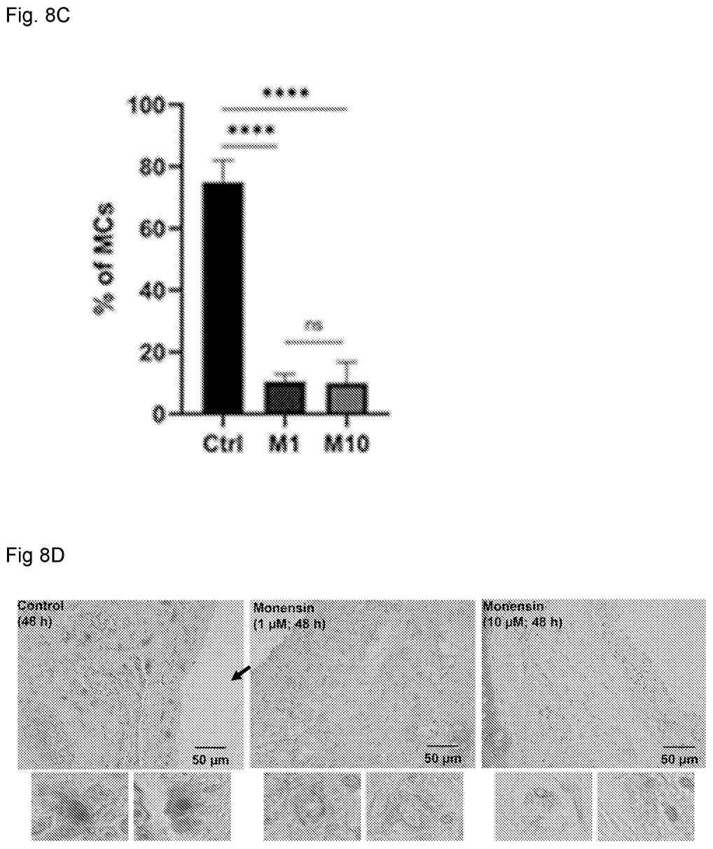

The data disclosed herein also show that monensin has a strong ability to prevent bronchoconstriction in an ex vivo setting of allergic airway inflammation (see FIGS. 8A-D).

Monensin induces apoptotic rather than necrotic cell death in mast cells (FIG. 2). From a therapeutic view, apoptosis is preferred over necrosis, considering that cellular necrosis may lead to the release of alarmins that might perpetuate inflammation, potentially causing tissue damage. The present data suggest a mechanism in which monensin triggers cell death by acting on the mast cell secretory granules, as supported by Example 4 (FIG. 4).

Altogether, these findings show that monensin is highly cytotoxic and selective to mature mast cells, and wherein the mast cell secretory granules are major targets for monensin.

The acidity of the granules was found to be a prerequisite for optimal responses to monensin. This was supported through experiments showing that the sensitivity of mast cells to monensin was reduced after blockade of proton pumping into the granules, showing that the granule acidity is a key factor in the execution of cell death. Further, the present data show that monensin causes a lowering of the intracellular pH, most likely as result of granule permeabilization leading to efflux of acidic granule contents to the cytosol (see Example 4, FIG. 4).

One likely explanation would be that the cytosolic acidification causes activation of pH-sensitive DNA:ses, a notion being in agreement with several previous studies. Typically, cell death in response to cytosolic acidification is known to occur in the absence of caspase activation and, in agreement with the latter, the present data show that monensin did not cause caspase 3 & 7 activation. Moreover, caspase inhibition did not prevent cell death in response to monensin (see FIG. 3). Altogether, a mechanism in which monensin causes granule permeabilization, which leads to decreased cytosolic pH, leads in turn to activation of caspase-independent apoptosis.

Hence, the present disclosure indicates that monensin induces cell death by targeting the mast cell secretory granules. Notably, mast cells have a markedly higher content of acidic granules than any other type of cells, and it is, thus, conceivable that monensin thereby shows selectivity for mast cells. Indeed, the data disclosed herein show that a range of other cell types were relatively refractory to monensin, and that monensin does not cause any gross lung tissue damage in an ex vivo setting.

However, one exception was eosinophils, which showed approximately equal sensitivity to monensin as did mast cells (FIG. 6E). Similar to mast cells, eosinophils have a high content of secretory granules, and it is therefore reasonable to assume that monensin acts on eosinophils by similar mechanisms as on mast cells, i.e., by permeabilization of their secretory granules.

Mast-dependent pathologies such as allergic asthma are typically accompanied by eosinophil infiltration, with eosinophils contributing to the pathology. Hence, the simultaneous targeting of both mast cells and eosinophils by monensin may be seen as a therapeutic advantage which could potentially lead to an effective intervention in pathogenic mechanisms by the combined action of MCs and eosinophils.

In summary, monensin has been shown to possess highly potent and selective anti-mast cell and anti-eosinophil properties at very low concentrations, making it highly useful as a drug for preventing, alleviating, abrogating, and treating inflammatory pathologies mediated by granulocytes comprising acidic cytoplasmic granules, such as mast cells and eosinophils, by inducing/causing mast cell apoptosis.

1.-21. (canceled).

22. A method for alleviating, preventing and/or treating a condition or disease mediated by granulocytes comprising acidic cytoplasmic granules in a subject in need thereof, comprising administering monensin to the subject.

23. The method according to claim 22, wherein said condition or disease involves activation of granulocytes comprising acidic cytoplasmic granules.

24. The method according to claim 22, wherein monensin selectively induces cell death in said granulocytes, wherein said cell death is apoptosis.

25. The method according to claim 22, wherein said granulocytes are mast cells and/or eosinophils.

26. The method according to claim 22, wherein said condition or disease is an inflammatory condition or disease.

27. The method according to claim 22, wherein said granulocytes are mast cells.

28. The method according to claim 27, wherein said condition or disease is selected from mastocytosis, mast cell activation syndrome, a type I hypersensitivity, cardiovascular disorders, hereditary alpha-tryptasemia, and cancer.

29. The method according to claim 27, wherein said condition or disease is mastocytosis.

30. The method according to claim 27, wherein said condition or disease is mast cell activation syndrome.

31. The method according to claim 27, wherein said condition or disease is a type I hypersensitivity.

32. The method according to claim 31, wherein said type I hypersensitivity is selected from allergic asthma, allergic rhinitis, allergic conjunctivitis, allergic dermatitis, anaphylaxis, urticarial, and angioedema.

33. The method according to claim 27, wherein the cardiovascular disorder is selected from atherosclerosis, cardiac fibrosis, and Kounis syndrome.

34. The method according to claim 22, wherein said granulocytes are eosinophils.

35. The method according to claim 34, wherein said condition or disease is selected from hypereosinophilic syndrome, eosinophilic gastrointestinal disorders, eosinophilic asthma, eosinophilic granuloma, eosionophilia-myalgia syndrome, Kimura disease, and angiolymphoid hyperplasia with eosinophilia.

36. The method according to claim 22, wherein the monensin is administered with a pharmaceutically acceptable carrier and/or excipient.

37. The method according to claim 22, wherein administration is performed by inhalation, oral, peroral, mucosal, or topical administration.

38. The method according to claim 22, wherein the monensin is administered in the form of a tablet, a powder, a gel, a capsule, an ointment, an aerosol, or a lyophilizate.

39. The method according to claim 22, wherein the monensin is administered in a pharmaceutically effective dose to the subject.

40. The method according to claim 22, wherein the monensin is administered with one or more additional anti-inflammatory therapies.

41. The method according to claim 22, wherein the monensin is administered in combination with one or more additional pharmaceutically active agents selected from the group consisting of antihistamines, glucocorticoids, adrenaline, sodium cromoglicate, nedocromil, montelukast, zafirlukast, mepolizumab, reslizumab, benralizumab, lebrikizumab, tralokinumab, dupilumab, omalizumab, tezepelumab, fevipiprant, and timapiprant.
